(19) United States
(12) Patent Application Publication
(10) Pub. No.: US 2013/0202637 A1
(43) Pub. Date: Aug. 8, 2013

(54) Dietary Supplements Including Ellagitannins and Ellagic Acid

(71) Applicant: John V. Cappello, King of Prussia, PA (US)

(72) Inventor: John V. Cappello, King of Prussia, PA (US)

(21) Appl. No.: 13/757,744

(22) Filed: Feb. 2, 2013

Related U.S. Application Data
(60) Provisional application No. 61/594,009, filed on Feb. 2, 2012.

(51) Int. Cl. A61K 36/05; A61K 45/06; A61K 36/03; A61K 31/352; A61K 31/137

(52) U.S. Cl. CPC A61K 36/05; A61K 31/352; A61K 31/137; A61K 36/03; A61K 45/06
USPC 424/195.17; 514/5.5; 514/453; 514/654

(57) ABSTRACT

A dietary supplement containing a source of ellagic acid, such as black raspberry seed powder, is combined with one or more of blue green algae, brown seaweed, phycocyanin and phenylathylemine. Additions of one or more vitamins and/or one or more minerals further improves the health enhancing benefits of the dietary supplement.

### CROSS REFERENCE TO RELATED APPLICATIONS

This application claims the benefit and priority of U.S. provisional patent application Ser. No. 61/594,009 entitled Production and Use of Concentrated Ellagic Acid, filed Feb. 2, 2012 and which is incorporated herein in its entirety.

### BACKGROUND AND SUMMARY OF THE DISCLOSURE

This disclosure is directed to a dietary composition which includes ellagitannins and/or ellagic acid, a process to create such a composition in concentrated form and to uses of the composition as a treatment and preventive for diseases such as cancer. Additional dietary supplements as well as vitamins, minerals, antioxidants, anti-inflammation ingredients and stem cell enhancement ingredients can be combined with ellagitannins to provide a composition for promoting health, quality of life and longevity.

Ellagitannins are polyphenolic antioxidants fanned from ellagic acid and a carbohydrate such as glucose. Abundant in fruits, seeds and nuts, ellagitannins are thought to have many protective properties on health, chief among them being potential anti-cancer properties. The metabolite of ellagitannins, ellagic acid, is formed during digestion. Ellagic acid when ingested directly is virtually destroyed in the digestive tract.

However, when ellagic acid is carried into the digestive tract by ellagitannins, the ellagic acid separates from the ellagitannins and enters the bloodstream in effective amounts. It is believed that the hydrolysis of ellagitannins facilitates the transport of ellagic acid into the bloodstream. While there are many sources of elagitannins, it would be beneficial to determine the best sources of ellagitannins, i.e. those being the most concentrated, effective or powerful for preventing and/or treating disease. In accordance with this disclosure, it has been found that the best or most concentrated source of ellagic acid is found in black raspberries.

Ellagic acid is a phenolic compound that has become known as a potent anti-oxidant compound. Ellagic acid itself is not thought to be naturally present in plants. Instead, polymers of gallic acid and hexahydroxydipenoyl (HHDP) are linked to glucose centers to form the class of compounds known as ellagitannins. When two gallic acid groups become linked side-by-side within a tannin molecule, an HHDP group is formed.

Ellagic acid results when the HHDP group is cleaved from the tannin molecule and spontaneously rearranges. Some articles in which ellagitannins are quantified refer to ellagic acid instead of ellagitannins because measuring of ellagitannins is done by breaking them down into ellagic acid subunits and quantifying the subunits.

Ellagic acid is a phytochemical or disease-fighting plant chemical that falls under the phenol classification, and offers powerful anti-cancer benefits according to the American Cancer Society. It acts as an antioxidant, enhancing immune function and protecting body cells from damage by free radicals, or highly reactive oxygen particles that come from the environment and pollution. It is believed that ellagic acid inhibits DNA mutations in cells that cause abnormal growth.

Ellagic acid is found in plant foods, especially red fruits and some nuts, and specifically in 46 different varieties of fruits and nuts. For example, ellagitannins are present in relatively high concentrations in red raspberries. In fact, red raspberries were believed to be the most concentrated source of ellagic acid.

Research on ellagic acid has focused heavily on its anticancer and antimicrobial properties. Ellagitannins are found in, for example, raspberries, blackberries, acai berries, pecans and walnuts. Studies have indicated that the main metabolite of ellagitannins (ellagic acid) is an effective anticancer agent. Its potential at preventing cancers of the cervix, colon, prostate, breast, esophagus, and skin have been studied. It is believed that many plants use ellagic acid to protect themselves against infection or destruction by germs and other organisms. Ellagitannins, and more specifically, their metabolite ellagic acid, is the reason acai berries, raspberries, and pomegranates have garnered their recent reputation as being “superfruits.”

When consumed, ellagitannins are rapidly metabolized to form ellagic acid and complementary carbohydrates. Researchers have found that when a variety of cancer cells are exposed to ellagic acid in a test tube, the ellagic acid disrupts the life cycle of cancer cells, usually by promoting cancer cell death but does not harm healthy cells. At least one study has shown ellagitannins may help reduce prostate-specific antigen (PSA) levels. Because ellagic acid is a relatively insoluble compound, there is some evidence that indicates it is not “bio-available” by direct ingestion.

By contrast, ellagitannins, the parent compounds of ellagic acid, are readily water-soluble, and researchers believe they may be more easily metabolized by the body, making them an excellent source for “delivering” ellagic acid to the body. This difference leads naturally to the conclusion that ellagic acid, consumed in the form of pure acid supplements, may be less effective than eating “real food” containing ellagitannins.

Ellagic acid can inhibit tumor growth in cancer of the skin, esophagus, lung and colon but it may be best known for offering protective benefits against breast and prostate cancer. In laboratory studies, ellagic acid reduces the effect of estrogen on promoting the growth of cancerous cells in breast tissue. Recent findings by the American Cancer Society also show that pomegranate juice significantly slows or even halts the increase of blood levels of prostate-specific antigen, or PSA, which is the marker used to test for prostate cancer as well as measuring the growth of prostate cancer. Protection was observed at a dose of 8 oz. of pomegranate juice a day.

Up until now, it was believed the richest food source of ellagic acid is the red raspberry. Other fruits considered to be excellent sources of ellagic acid are strawberries, cranberries, grapes, blackberries, blueberries, cherries, pomegranates and pomegranate juice. Apples also offer some ellagic acid. Other plant sources of ellagic acid include nuts. Varieties of nuts that offer appreciable amounts of ellagic acid include walnuts and pecans.

Each source of ellagitannins has its own individual mix of ellagitannins. One way to think about this is that the active ingredients in both green tea and grape seeds is a group of bio-chemicals called polyphenols (the same group of bio-chemicals that includes ellagic acid)—but because the mix of polyphenols is different in green tea and grape seeds, they each have an entirely different range of effects on the human body. Of the different sources of ellagitannins, the only “mix” that has been verified effective is the mix in red raspberries. Red raspberries are currently believed to be the most proven source of ellagic-acid-producing ellagitannins, with pomegranates probably a good alternative.

Consuming one cup of red raspberries per day (providing 40 mg of ellagitannins) can prevent the development of cancer cells. At low concentrations, ellagic acid slows the growth of cancer cells; at higher concentrations, it causes cancer cells to die. For example, cells infected with the human papilloma virus (which is linked to cervical cancer), when exposed to ellagic acid experienced apoptosis, or normal cell death.

Red raspberry ellagitannins slow the growth of abnormal colon cells in humans. Tests reveal similar results for breast, pancreas, esophageal, skin, and prostate cancer cells. The ellagitannins also produce a breakdown in human leukemia cells.

Red raspberry ellagitannins have been shown to prevent destruction of the P53 gene by cancer cells. P53 prevents mutagenic activity in cervical cells. Red raspberry ellagitannins also protect human cells against cancer-causing agents in tobacco smoke, food additives, and petroleum-based substances. Ellagitannins act as scavengers to “bind” cancer-causing chemicals, making them inactive. The ellagitannins inhibit the ability of other chemicals to cause mutations in bacteria. Red Raspberry ellagitannins also protect DNA by blocking carcinogens from binding to the DNA.

Another way that ellagitannins protect the body is through G-Arrest, whereby the ellagitannins stop cells from mutating in the first place. If cells don't mutate, then cancer is avoided. This is very important when one considers that every single day a human body produces anywhere from a few hundred to as many as 10,000 cancerous cells as part of normal metabolism. In tests conducted with ellagic acid, G-Arrest began in 72 hours in both breast and cervical cancer.

European medical studies also demonstrate that red raspberry ellagitannins lower the incidence of birth defects, promote wound healing, reduce heart disease, and may reduce or reverse chemically induced liver fibrosis. In addition, the ellagic acid produced from the ellagitannins has anti-bacterial and anti-viral properties

Most of the above studies have been conducted in two phases. First, in test tubes on isolated cells using ellagic acid. Later (and ongoing, even now) in live human beings using the full ellagitannin complex as found in red raspberry puree. It is, of course, the human studies that are of interest.

The Meeker variety of red raspberry is the best currently known source of ellagic acid followed by Chilliwack and Willamette varieties of red raspberry. The amount of ellagic acid derived from three different varieties of red raspberries is shown in Table 1.

TABLE 1
The amount of ellagic acid from raspberries.
Ellagic acid (micrograms/gram dry weight)

| Variety | Seeds | Pulp | Whole |
|---|---|---|---|
| Meeker | 8.4 | 3.36 | 4.31 |
| Chilliwack | 7.78 | 2.58 | 3.39 |
| Willamette | 8.13 | 2.05 | 2.91 |
| Average | 8.1 | 2.66 | 3.54 |

The availability to the body of ellagic acid from dietary sources has only been confirmed with red raspberries. Other foods such as strawberries, pomegranates, and walnuts contain far lesser amounts ellagic acid and their bioavailability has not been confirmed.

### DETAILED DESCRIPTION OF REPRESENTATIVE EMBODIMENTS

Although a review of all current information relevant to ellagic acid suggests red raspberry seed is the best source for providing a natural cancer killing agent, additional research conducted under this disclosure has discovered an even more concentrated source of ellagic acid. This source is black raspberry, and in particular, black raspberry seeds. In fact, the amount of ellagic acid available from black raspberry seeds is sufficiently concentrated to allow for a reduction in the amount of raspberry seed by up to 50% or more as compared to red raspberry seed while providing the same amount of ellagic acid.

While effective amounts of ellagic acid can be obtained by eating large amounts of fruits and/or nuts, it is more practical and convenient to be able to provide concentrated amounts of ellagic acid in the form of ellagitannin in pills, tablets or capsules.

In order to concentrate the ellagitannin in various fruits or nuts, the seeds of the fruits or even the fruit itself can be pulverized in such a way so as to create nano particles and provide the most available surface area for facilitating complete digestion and entry into the bloodstream. A slurry of these seeds can be made with distilled water. This slurry can be heated to a temperature below boiling to capture the water soluble components. The heating should be for one half to one hour, and then let to cool until at ambient temperature.

The post heated and cooled slurry should be filtered to obtain all of the water soluble elements of composition which would include elligitannins as well as polyphenols and ellagic acid. Intrinsic to this filtrate are simple and complex saccharide molecules. The resultant retained filtrate material can be analyzed for phytochemical content and for any other commercial use. The resultant filtered broth should then be isolated by freeze drying, evaporation, spray drying or any other commercially feasible method of isolation.

The isolated powder filtrate from the broth indicated above can be made into drinks, capsules, tablets or any other form suitable for oral ingestion. The filtrate serves as a targeted cancer killer with cancer cells being heavily attracted to its saccharides and glucosides and bringing with it the water soluble elligitannins, polyphenols and particularly ellagic acid.

The filtrate can be used as a single cancer treatment nutraceutical or drug, used in combination with other cancer treatments, or used as a cancer prevention aid by offering immune system enhancement. Numerous other benefits could arise from ingestion of these filtrate concentrations.

As noted above, it has been discovered that black raspberry seeds contain the highest concentrations of ellagic acid. These seeds can be processed as described above or simply ingested whole, in powder form in a slurry or tea, or any other suitable form for ingestion. Whole, pureed, or otherwise processed black raspberries can also be ingested for delivering ellagic acid to the bloodstream via digestion of ellagitannins.

While looking for the most concentrated source of ellagic acid based on endogenous ellagic acid release, black raspberry seed powders were compared to red raspberry seed powders. The results of this study are shown in Table 2 below. The samples in column 1 were obtained from four different commercial sources of red and black raspberries.

TABLE 2

| Sample Source | Total Ellagic Acid mg/gram |
|---|---|
| 1 Red Raspberry Seed Powder | 7.06 |
| 1 Black Raspberry Seed Powder | 16.65 |
| 2 Red Raspberry Seed Powder | 7.63 |
| 2 Black Raspberry Seed Powder | 12.04 |
| 3 Red Raspberry Seed Powder | 8.11 |
| 3 Black Raspberry Seed Powder | 11.5 |
| 4 Red Raspberry Seed Powder | 2.27 |
| 4 Red Raspberry Seed Powder | 1.48 |

When ingested, raspberry fruit or seeds act as a sort of “Trojan Horse” to aid in the destruction and control of cancer cells. In other words, the cancer cells are attracted by the polysaccharides or sugars contained in the fruit or seed coupled with the ellagitannins. The ellagitannins then break down within the cancer cell and produce ellagic acid which is not toxic to normal cells, but is toxic to cancer cells.

Many of the studies in the literature, including the Hollings Cancer Institute study, suggest as little as 50 mg. of ellagic acid endogenously released from ellagitannin complex have been found beneficial in both human and test tube studies in destruction of cancer cells. Based on the values of sample source one in Table 2 above, the total ellagic acid of 16.65 mg. per gram of black raspberry seed powder is the most concentrated source of ellagic acid known to date. Three hundred grams of this powder would contain about 4995 mg. of ellagic acid. If one were to break this down to servings per day based upon 30 10 gram doses, each dose would provide up to 166.5 mg. of endogenous ellagic acid per serving. This is more than three times the minimum effective amount of 50 mg.

A nutritional supplement containing 300 grams of such a source of black raspberry seed powder or any other source of ellagic acid as designated would offer further efficacy by its greater concentrations for cancer cell death.

While ingesting a source of ellagic acid promotes health by providing a strong antioxidant with cancer fighting properties, even greater beneficial health effects can be achieved by combining a source of ellagic acid, such as in any of the forms and sources disclosed above, with a dietary supplement providing additional nutritional benefits. That is, those fighting cancer typically lose body weight due to cancer cells consuming large amounts of nutrition which otherwise would be consumed by healthy cells. This can cause a wasting away. By providing a source of ellagic acid in combination with a nutritional supplement, the detrimental loss of body weight can be reduced and a generally more healthy condition can be maintained to allow a person to more effectively fight a disease such as cancer.

In those people who are not fighting cancer, the combination of a source of ellagic acid and a nutritional supplement can help to avoid the onset of cancer. As noted above, the human body produces many cancer cells every day. By maintaining a healthy, nutritionally sound body with cancer fighting supplements, the development and spread of cancer cells can be minimized.

A further health benefit can be achieved by adding one or more vitamins and/or one or more minerals to a source of ellagic acid alone, or to a source of ellagic acid in combination with one or more nutritional supplements as noted above.

Yet further health benefits can be achieved by combining a source of ellagic acid with a nutritional or dietary supplement that promotes and stimulates the body's natural production of stem cells. By increasing the number of stem cells in circulation and by increasing their ability to differentiate, the body can repair itself more efficiently. This a great benefit when the body is fighting a disease such as cancer.

For example, as disclosed in U.S. Pat. No. 7,473,427, which is incorporated herein by reference in its entirety, a dietary supplement which can stimulate stem cell growth includes a blue green algae such as Aphanizomenon flos-aqua and concentrated extracts of whole blue green algae. These extracts include concentrates of phycocyanin and phenylethylamine.

In one example, at least about 250 mg of whole blue green algae is combined with at least about 25 mg of phycocyanin and at least about 0.35 mg of phenylethylamine in a tablet, capsule or drink. It should be noted that phycocyanin can be provided in the form of Arthrospira platensis instead of an extract of blue green algae. Moreover, whole blue green algae can be supplemented with or replaced with a brown seaweed, such as fucoidan. From 50 to 150 mg of fucoidan can replace whole blue green algae in the composition above. This combination of nutritional supplements can stimulate the production of stem cells and improve their “plasticity” or ability to differentiate into different types of body cells. This formulation of nutrients is currently available commercially under the brand “VitaStim.”

The whole blue green algae (and/or fucoidan), phycocyanin and phenylethylanine can be combined in the amounts noted above with a source of ellagic acid such as any of the sources of ellagitannins disclosed above to provide the health benefits noted above. For example, 10 grams of black raspberry seed powder obtained from the Fruit Smart Company containing 16.65 mg/gram of total ellagic acid would provide about 166.5 mg of endogenous ellagic acid per serving.

Additional health benefits can be achieved, as noted above, by combining a source of ellagic acid with one or more vitamins and/or one or more minerals. As disclosed in U.S. Pat. No. 8,334,131, which is incorporated by reference herein in its entirety, adding from about 6.25 to 125 mg of vitamin D3 to the blue green algae formulation disclosed above improves stem cell circulation within the human body.

Moreover, by adding from at least about 15 mg to several grams of vitamin C to the blue green algae formulation disclosed above, the ability of stem cells to differentiate within the human body is enhanced. Adding vitamins D3 and C to a source of ellagic acid can enhance and promote health by improving stem cell circulation and plasticity. By further adding blue green algae, brown seaweed, phycocyanin and phenylethylamine to the combination of a source of ellagic acid and one or more vitamins, such as vitamins D3 and C, an effective disease fighting and health promoting composition results.

Additional vitamins can add additional health benefits to the health promoting composition of a source of ellagic acid, blue green algae and/or brown seaweed, such as fucoidan, phycocyanin, phenylethylamine and vitamins D3 and C. Additional vitamins include vitamins A, E, K, thiamin, riboflavin, niacin, vitamin B6, folic acid and pantothenic acid.

Minerals such as calcium iron, phosphorus, iodine, magnesium, zinc, selenium, copper, manganese, chromium and molybdenum can also be combined with any source of ellagic acid to further promote health. These minerals can also be combined with any source of ellagic acid and any one or more of blue green algae, brown seaweed (such as fucoidan), phycocyanin, phenylethylamine, and one or more vitamins as described above.

To further promote health and fight disease such as cancer additional minerals such as one or more of boron, selenium, silicon, chloride, sodium, tin, cobalt, titanium, nickel, vanadium, fluoride, potassium and germanium can be combined with a source of ellagic acid or with any of the other combinations described above.

Representative daily dosages of the vitamins and minerals combinable with one or more of a source of ellagic acid, blue green algae, brown seaweed, phycocyanin, and phenylethylamine are disclosed in U.S. Pat. No. 8,334,131.

A commercially available source of a formulation of blue green algae, phycocyanin d phenylethylamine can be obtained from Emergent Health Corporation (www.emergenthealth.com) of King of Prussia, Pa. under the brand “Vita-Stim.” Combining one or more Vita-Stim capsules with a source of ellagic acid such as black raspberry seed powder provides the health benefits noted above.

Even greater health benefits can be achieved by combining a source of ellagic acid such as black raspberry seed powder with a combination of Vita-Stim constituents, namely, blue green algae, phycocyaniun and phenylathylamine, and with one or vitamins and minerals. Such a combination of Vita-Stim constituents and vitamins and minerals is available commercially from Emergent Health Corporation under the brand “JD1 Multivitamin/Multimineral. This formulation contains the ingredients in the amounts identified in U.S. Pat. No. 8,334,131.

In one example, ten grams of black raspberry seed powder can be combined with one or more Vita-Stim capsules dispersed in water or shaken in water, or in any fruit juice or other medium tolerable for oral ingestion. Alternatively, a source of ellagic acid such as black raspberry seed powder can be combined with the Vita-Stim constituents and made into capsules, tablets or any other ingestible format such as puddings, gelatins, smoothies and the like. Another example is simply substituting the JDI Multivitamin/Multimineral for the Vita-Stim constituents noted in the example immediately above.

Of course these commercially available supplements could likewise be combined with a source of ellagic acid, such as black raspberry seeds, to provide an effective disease fighting and health promoting composition.

It will be appreciated by those skilled in the art that the above dietary supplements including ellagitannins and ellagic acid is merely representative of the many possible embodiments of the disclosure and that the scope of the disclosure should not be limited thereto.

What is claimed:

1. A dietary supplement, comprising:
a source of ellagic acid; and
one or more of the group consisting of a blue green algae, a brown seaweed, phycocyanin and phenylethylamine.

2. The supplement of claim 1, further comprising one or more supplemental vitamins.

3. The supplement of claim 1, further comprising one or more supplemental minerals.

4. The supplement of claim 1 wherein said source of ellagic acid comprises raspberries.

5. The supplement of claim 4, wherein said raspberries comprise black raspberries.

6. The supplement of claim 1, wherein said source of ellagic acid comprises raspberry seed powder.

7. The supplement of claim 6, wherein said raspberry seed powder comprises black raspberry seed powder.

8. A dietary supplement comprising:
a source of ellagic acid;
at least one of the group consisting of a blue green algae and a brown seaweed;
phycocyanin; and
phenylethylamine.

9. The supplement of claim 8, wherein said blue green algae comprises Aphanizomenon flos-aqua and said brown seaweed comprises fucoidan.

10. The supplement of claim 8, wherein said phycocyanin is present as a component of Arthrospira platensis.

11. The supplement of claim 8, further comprising a supplemental vitamin selected from the group consisting of vitamin C and vitamin D3.

12. The supplement of claim 8, further comprising one or more supplemental vitamins.

13. The supplement of claim 12, wherein said one or more supplemental vitamins is selected from the group consisting of vitamin A, vitamin C, vitamin E, vitamin K, thiamin, riboflavin, niacin, vitamin B6, folic acid, vitamin B12, biotin and pantothenic acid.

14. The supplement of claim 8, further comprising one or more supplemental minerals.

15. The supplement of claim 14, wherein said one or more supplemental minerals is selected from the group consisting of calcium, iron, phosphorous, iodine, magnesium, zinc, selenium, copper, manganese, chromium and molybdenum.

16. The supplement of claim 8, wherein said source of ellagic acid comprises raspberries.

17. The supplement of claim 8, wherein said source of ellagic acid comprises a concentrated source of ellagic acid comprising a powder formed by heating a slurry, cooling the slurry and filtering the slurry to obtain a filtrate comprising said powder.

18. The supplement of claim 8, wherein said source of ellagic acid comprises raspberry seeds.

19. The supplement of claim 8, wherein said source of ellagic acid comprises black raspberry seed powder.

20. A method of improving health and resisting disease, comprising:
orally administrating to a person a source of ellagic acid comprising black raspberry seed powder, and one or more of the group consisting of blue green algae and brown seaweed.
